(19) United States
(12) Patent Application Publication
(10) Pub. No.: US 2007/0113855 A1
(43) Pub. Date: May 24, 2007

(54) Respiratory apparatus with improved seal

(75) Inventor: Wayne D. Carlsen, Riverton, UT (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/283,441

(22) Filed: Nov. 18, 2005

(51) Int. Cl. A62B 18/10; A61M 5/315; A61M 5/31; A61M 25/16; A61M 5/00; A61M 25/18

(52) U.S. Cl. 128/207.12; 604/236; 604/243; 604/538

(57) ABSTRACT

A respiratory apparatus includes a seal disposed within a port around a suction catheter so as to provide a sliding frictional sealing fit with the suction catheter. The seal is formed from a resilient material and is shaped to contain a radially outer flange section, an inner conically shaped skirt section defining an aperture through which the suction catheter passes, and a bridge section between the outer flange section and the conical skirt section. The outer flange section defines an inner diameter and the bridge section extends radially inward from the inner diameter at a first angle relative to the outer flange section. The conical skirt section extends radially inward from the bridge section at a second angle relative to the bridge section that is less than the first angle and a seal lip is defined at an end of the conical skirt section.

### BACKGROUND

A variety of different circumstances exist in which a person may be required to have an artificial airway, such as an endotracheal tube, placed in their respiratory system. During surgery, for instance, the artificial airway functions to keep the patient's airway open so that adequate lung ventilation is maintained during the surgical procedure. Alternatively, with many patients the endotracheal tube will remain in place to sustain mechanical ventilation for a prolonged period.

If an endotracheal tube is to be left in place for any substantial amount of time, it is critical that respiratory secretions be periodically removed. This is usually accomplished with the use of a respiratory suction catheter. As the suction catheter is withdrawn, a negative pressure may be applied to the interior of the catheter to draw mucus and other secretions from the respiratory system.

With conventional closed suction catheter assemblies, the catheter tube is enveloped by a protective sleeve. The catheter assembly includes a valve mechanism in communication with a vacuum source to control the suctioning process. At its distal patient end, the closed suction catheter assembly is attached to a manifold, connector, adaptor, or the like.

After the application of negative pressure, the catheter tube may be withdrawn from the artificial airway and, as the catheter tube is pulled back into the protective sleeve, a resilient wiper or seal within the distal end manifold strips or scrapes a substantial portion of any mucus or secretions from the outside of the catheter tube. The seal also prevents the patient's ventilation air from escaping from around the suction catheter.

The current seal design is a flat, washer-shaped disc with the inner diameter of the disc generating a sliding friction fit with the catheter. This friction fit must be sufficient to ensure a proper wiping action upon withdrawal of the catheter as well as prevent the escape of ventilation air and, in this regard, a relatively tight fit is necessary between the catheter and the seal. This requirement often results in difficulty in sliding the catheter through the seal, particularly for insertion into the patient's airway, and may result in stenosis or necking-down of the catheter diameter. Moreover, this configuration provides little tolerance for catheter diameter variances.

Thus, a need exists in the art for an improved wiper or seal in a respiratory suction apparatus that addresses these drawbacks with conventional seal designs.

### SUMMARY

Various objects and advantages of the invention will be set forth in part in the following description, or may be apparent from the description, or may be learned from practice of the invention.

A respiratory apparatus according to the invention includes a suction catheter having a tubular portion with a lumen defined therethrough. The suction catheter is adapted for removing fluids from a patient by insertion of the tubular portion into a patient's artificial airway with subsequent application of negative pressure to the proximal end of the lumen. A manifold is configured in communication with the patient's artificial airway and includes a port through which the suction catheter is advanced and withdrawn from the patient's artificial airway.

A resilient material seal is disposed within the manifold port and includes an aperture through which the suction catheter passes. This seal provides a sliding frictional sealing fit with the suction catheter and serves to wipe or scrape respiratory secretions from the outer surface of the suction catheter as the catheter is withdrawn from the patient's airway.

In accordance with certain aspects of the invention, the wiper seal has a unique configuration that provides the seal with distinct advantages. In a particular embodiment, the seal includes a radially outer flange section and an inner conically shaped skirt section defining the aperture through which the suction catheter passes. A bridge section is provided between the outer flange section and the conical skirt section. The outer flange section defines an inner diameter with the bridge section extending radially inward from this inner diameter at a first angle relative to the outer flange section. The conical skirt section extends radially inward from the bridge section at a second angle relative to the bridge section that is less than the first angle between the bridge section and the outer flange. A seal lip is configured at the end of the conical skirt section to engage against the outer surface of the suction catheter.

The angled configuration between the various sections of the seal, selection of material, and relative thickness of the sections all contribute to provide the seal with unique characteristics. For instance, when the suction catheter is slid through the seal as the catheter is advanced into the patient's airway, frictional resistance between the conical skirt section (the sealing lip in particular) is reduced as compared to conventional washer-type seals without sacrificing seal effectiveness. Resistive forces are directed longitudinally along the length of the skirt section such that the skirt section tends to stretch along this axis. Also, the angle between the skirt section and bridge section defines a flex point between the sections that allows the skirt section to flex radially outward towards the outer flange section. A flex point is also defined between the bridge section and outer flange section. These features allow the seal to effectively accommodate a wider range of suction catheter diameters as compared to conventional seal designs.

In a particular embodiment, the outer flange section has a radial thickness greater than a thickness of the conical skirt section. The outer flange section may also have a radial thickness greater than the thickness of the bridge section. This configuration may be desired to enhance the flex action between the bridge section and outer flange section. In an alternate embodiment, the bridge section and conical skirt section may have generally about the same thickness.

In a particularly unique embodiment, the outer flange section has a radial thickness greater than the thickness of the conical skirt section and the bridge section, and the bridge section has a thickness greater than the thickness of the conical skirt section.

The first angle between the bridge section and the outer flange section may vary. For example, in one embodiment this angle may be about 90 degrees such that the bridge section extends essentially perpendicular to the flange section. In an alternate embodiment the first angle may be greater than 90 degrees.

The second angle between the bridge section and the conical skirt section may also vary, and may be a function of the first angle between the bridge section and flange section. For example, the second angle will generally greater than 90 degrees when the first angle is 90 degrees or less. If the first angle is greater than 90 degrees, the second angle will generally be 90 degrees or less.

Although not a requirement, the bridge section may be defined at a longitudinal end of the outer flange section. In an alternative embodiment, the outer flange section may extend longitudinally on either side of the bridge section.

The seal may be made of various known resilient seal materials, with all of the seal sections being integrally formed into a single seal component.

The seal lip may have various configurations, and may be directly formed with the conical skirt section or subsequently defined at the end of the skirt section.

In a particular embodiment, the seal lip is a relatively sharp point edge defined by angled planar surfaces at the end of the conical skirt section. This configuration provides a point knife-edge engagement against the catheter surface. In an alternate embodiment, the seal lip is a planar surface configured to engage the catheter along a longitudinally extending plane. In still another embodiment, the seal lip may be provided with a rounded edge.

Aspects of the invention will be described in greater detail below by reference to particular embodiments illustrated in the figures.

### BRIEF DESCRIPTION OF THE DRAWINGS

FIG. 1 is a perspective view of a respiratory apparatus that may incorporate a seal in accordance with the invention.

FIG. 2 is a cross-sectional view of the manifold section of the respiratory apparatus that incorporates a seal in accordance with the invention.

FIG. 3A is cross-sectional view of the seal position and orientation within the manifold prior to insertion of suction catheter through the seal.

FIG. 3B is a cross-sectional view of the seal in FIG. 3A after insertion of the suction catheter through the seal.

FIG. 4 is a cross-sectional view of an alternate embodiment of a seal configuration in accordance with the invention.

FIG. 5A is a cross-sectional view of another alternate embodiment of a seal configuration in accordance with the invention.

FIG. 5B is a cross-sectional view of still another embodiment of the seal configuration in accordance with the invention.

### DETAILED DESCRIPTION

Reference will now be made in detail to embodiments of the invention, one or more examples of which are illustrated in the drawings. Each example is provided by way of explanation of the invention, and is not meant as a limitation of the invention. For example, features illustrated or described as part of one embodiment may be used with another embodiment to yield still a third embodiment. It is intended that the present invention include these and other modifications and variations.

As used herein, “proximal” refers generally to the direction towards a medical caregiver. “Distal” refers generally to the direction towards a patient.

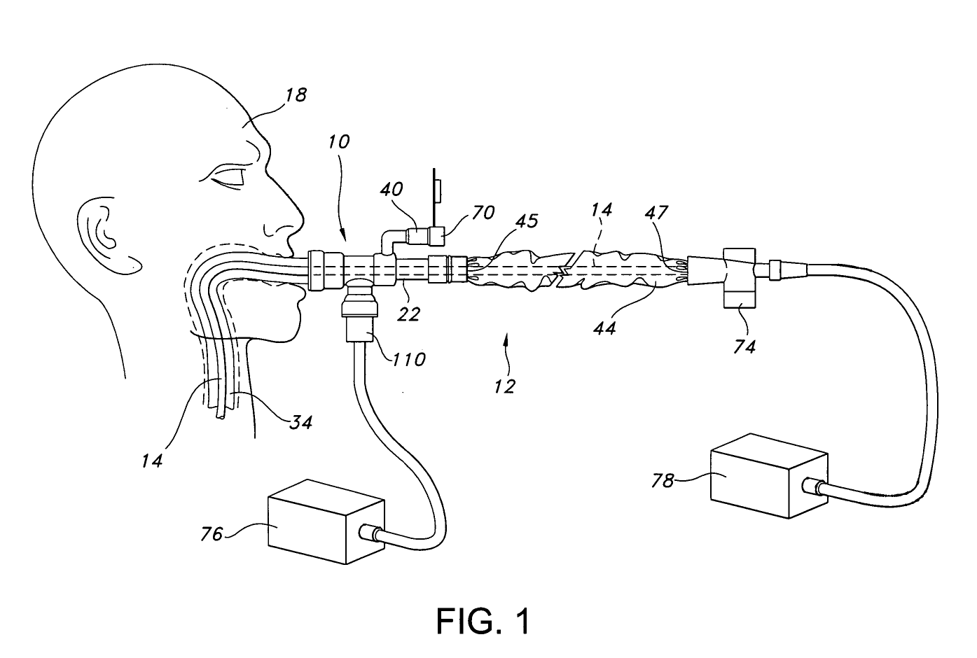

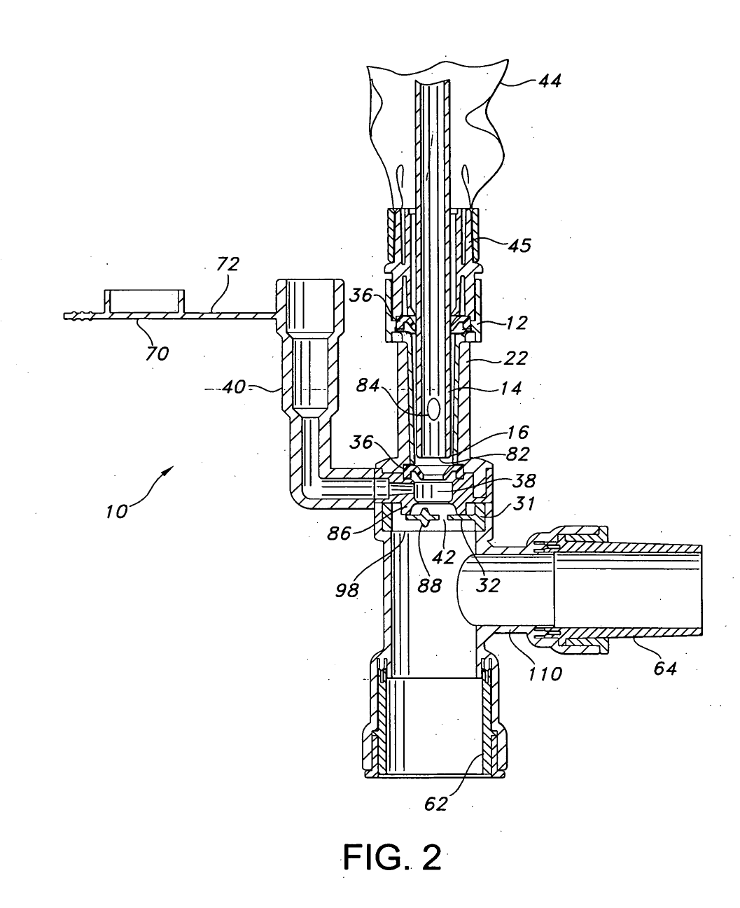

The present invention relates to a respiratory apparatus that connects to a patient's artificial airway for a variety of purposes. Referring to FIG. 1, an embodiment of the respiratory apparatus 10 is depicted generally as it would be connected to the artificial airway 34 of a patient 18. The apparatus 10 includes a suction catheter 12 and related components. A ventilator 76 may be in communication with the artificial airway 34 through a swiveling port 64 (FIG. 2) of a manifold 110. The manifold 110 includes swiveling port 62 (FIG. 2) for connection to the airway 34. The ventilator 76 may provide air to and remove air from the lungs of the patient 18 through the artificial airway 34.

It is to be understood that the configuration of the manifold 110 shown in FIGS. 1 and 2 is only an exemplary embodiment of the present invention, and the present invention is not limited to such a manifold. The respiratory apparatus 10 in accordance with the present invention may be provided with manifolds 110 of any configuration known in the art.

If the artificial airway 34 is left in the patient 18 for any substantial amount of time, respiratory secretions may build up in the lungs of the patient 18. As such, these secretions should be removed in order to ensure that adequate lung ventilation of the patient 18 is maintained. These secretions may be removed through use of the suction catheter 12. The suction catheter 12 has a tubular portion 14 having a distal end 16 with a distal opening 82 therein and a side opening 84 (FIG. 2) that may be extended through the artificial airway 34 into the lungs of the patient 18. A vacuum source 78 may be in communication with the ventilating circuit, and more specifically in communication with the suction catheter 12. A medical caregiver actuates a suction valve 74 to apply vacuum pressure to the tubular portion 14 of the suction catheter 12. Upon doing so, respiratory secretions in the patient 18 and in the artificial airway 34 may be removed.

Respiratory secretions may sometimes remain on the tubular portion 14 of the suction catheter 12 or transfer onto other portions of the ventilator circuit. These respiratory secretions are undesirable in that they provide a breeding ground for pathogens and other harmful agents that may harm the patient 18. It is therefore the case that the suction catheter 12 and/or other components of the ventilation circuit may be cleaned in order to remove any residual respiratory secretions. In order to ensure a lower risk of contamination to the patient 18, it is common practice to remove and replace the suction catheter 12 after some amount of set time has passed, for instance after 24 or 72 hours of use.

As may be seen in FIG. 2, the suction catheter 12 is shown with a flexible plastic sleeve 44. The sleeve 44 may be present in order to contain and isolate respiratory secretions that accumulate on the tubular portion 14 of the suction catheter 12 as the tubular portion 14 is withdrawn from the ventilation circuit.

The sleeve 44 may be provided on either end with sealing connections 45 and 47 that attach the sleeve 44 to the suction catheter 12.

The manifold 110 may be permanently attached to the suction catheter 12 and detachable from the artificial airway 34 so that a new suction catheter 12 may be incorporated into the ventilation circuit. In an alternate embodiment, the suction catheter 12 may be removably attached to the manifold 110, which remains attached to the artificial airway. This embodiment is described in detail in co-pending and commonly owned U.S. application Ser. No. 10/430813 filed on May 6, 2003 incorporated herein by reference for all purposes.

The respiratory apparatus 10 includes an instrument introduction section 22 having a passageway extending therethrough. The tubular portion 14 of the suction catheter 12 is advanced through this passageway, through an opening 98 and into the manifold 110, and eventually advanced into the artificial airway 34 (FIG. 1). Upon retraction of the tubular portion 14 from the patient 18, respiratory secretions may be present on the surface of the tubular portion 14. At least one wiper seal 36 may be provided in the instrument introduction section 22. FIG. 2 depicts an upper and a lower wiper seal 36, the lower wiper seal 36 may be provided on some suction catheters, but is in addition to the upper wiper seal 36.

Various embodiments of the wiper seal 36 in accordance with aspects of the invention will be described in detail below. In general, the wiper seal 36 is a resilient member having an aperture through which the tubular portion 14 passes. The wiper seal 36 frictionally engages the tubular portion 14 as the tubular portion 14 is retracted from the artificial airway 34 to a position proximal from the wiper seal 36. Respiratory secretions present on the surface of the tubular portion 14 are removed by the sliding frictional engagement between the wiper seal 36 and tubular portion 14.

Referring to FIG. 2, the instrument introduction section 22 may also be provided with a cleaning section 38. In one exemplary embodiment, the cleaning section 38 may be defined by a cleaning section member 86. Additionally or alternatively, the cleaning section 38 may be defined on one end by the upper surface of a valve 32 and the lower surface of the upper wiper seal 36 located proximal to the sealing connection 45. The valve 32 is shown in a closed position in FIG. 2 as a single flap that is hingedly attached to an annular ring 31 housed within the instrument introduction attachment section 22. The hinge on the valve 32 may provide both a bias force and a pivoting location. The valve 32 may be opened by insertion of the tubular portion 14 through the instrument introduction section 22. A projection 88 on the valve is configured to minimize contact of the valve with the surface of the tubular portion 14. The valve 32 may include an aperture 42 that helps to establish a more desirable turbulent fluid flow with the cleaning section 38. Use of such a valve 32 is disclosed in U.S. Pat. 6,227,200 B1issued to Crump et al., the entire disclosure of which is incorporated by reference herein in its entirety for all purposes.

The tubular portion 14 of the suction catheter 12 may be cleaned by positioning the distal end 16 of the suction catheter 12 proximal to wiper seal 36. Upon so positioning, a vacuum may be drawn through the suction catheter 12 and lavage or other cleaning solution may be injected into the cleaning section 38. Application of vacuum may cause the valve 32 to be forced against the distal side of cleaning section member 86 and form the distal end of cleaning section 38. However, in other exemplary embodiments, the valve 32 may be biased with enough force to close and seal against the distal side of cleaning section member 86 without application of suction force. However, it is to be understood that injection of lavage or other cleaning solutions and/or application of a vacuum may be performed in instances not associated with cleaning of the tubular portion 14 of the suction catheter 12.

An irrigation port 40 may be provided with the instrument introduction section 22 in order to allow for the injection of the lavage solution. A container (not shown) holding the lavage solution may have an outlet inserted into the irrigation port 40. Lavage may then be dispensed from this container into the irrigation port 40 which may be in communication with the cleaning section 38. The irrigation port 40 may also be provided with an irrigation cap 70 that may be connected to the irrigation port 40 by way of a tether 72. The irrigation cap 70 may be placed onto the irrigation port 40 in order to close the irrigation port 40 when not in use.

In certain exemplary embodiments of the present invention, the cleaning section member 86 may be configured such that a small amount of space is present between the tubular portion 14 of the suction catheter 12 and the cleaning section member 86. In certain exemplary embodiments of the present invention, this space may be between about 0.005 and about 0.015 inches. This space provides two advantages. First, if lavage is needed to be provided to the patient 18, injection of lavage through the irrigation port 40 and then into the cleaning section 38 causes a stream of lavage solution to be directed out of the manifold 110 and into the patient 18. Second, as the tubular portion 14 is withdrawn, the close proximity between the tubular portion 14 and the cleaning section member 86 may help to wipe any heavy layers of respiratory secretions from the outside of the tubular portion 14 of the suction catheter 12.

As shown in FIG. 2, the instrument introduction section 22 is in axial alignment with the swiveling port 62 that may be further attached to the artificial airway 34 (FIG. 1). This alignment may help to reduce contamination due to the fact that the suction catheter 12 during withdrawal from the patient 18 (FIG. 1) does not have to pass around bends or over other objects in order to be withdrawn into the instrument introduction section 22 proximate to the valve 32. In effect, this arrangement is a “straight shot” that reduces the chances of respiratory secretions being scraped off of the tubular portion 14 of the suction catheter 12 and being deposited onto a bend or other obstacle in the respiratory apparatus 10.

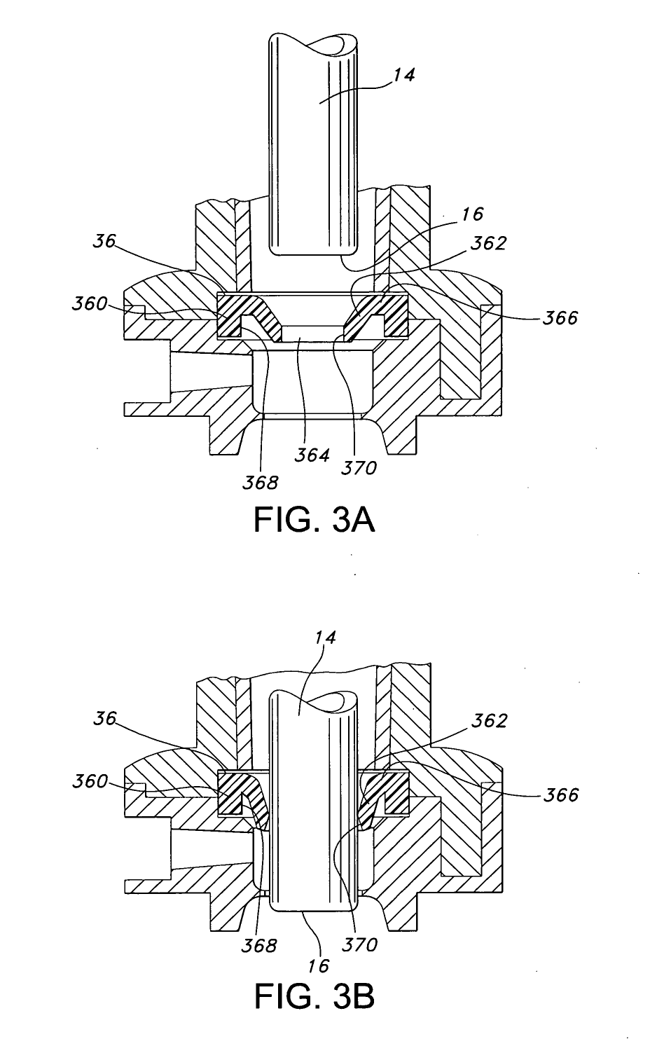

Turning now to FIGS. 3A and 3B, some constructions of the seal 36 are depicted. As shown, the seal 36 is disposed within the port between the manifold 110 and the instrument introduction section 22. In one possible embodiment, the seal 36 includes a radially outer flange section 360 and an inner conically shaped skirt section 362 defining an aperture 364 through which the suction catheter 12 passes. A bridge section 366 is provided between the outer flange section 360 and the conical skirt section 362. The outer flange section 360 defines an inner diameter 368 with the bridge section 366 extending radially inward from this inner diameter 368 at a first angle relative to the outer flange section 360. The conical skirt section 362 extends radially inward from the bridge section 366 at a second angle relative to the bridge section that is less than the first angle between the bridge section and the outer flange 360. A seal lip 370 is configured at the end of the conical skirt section 362 to engage against the outer surface of the suction catheter.

In accordance with certain aspects of the invention, the wiper seal 36 has a unique configuration which provides it with distinct advantages. These features include an angled configuration between the various sections of the seal 36, the selection of material, and the relative thickness of each of the sections 360, 362, and 366. All contribute to provide the seal with unique characteristics. For instance, when the suction catheter 12 is slid through the seal 36 as the catheter is advanced into the patient's airway as shown in FIG. 3B, frictional resistance between the conical skirt section 362 (the sealing lip 370 in particular) is reduced as compared to conventional washer-type seals. This is accomplished without sacrificing seal effectiveness. Resistive forces are directed longitudinally along the length of the skirt section 362 such that the skirt section tends to stretch along this axis. Also, the angle between the skirt section 362 and bridge section 366 defines a flex point between the sections that allows the skirt section 362 to flex radially outward towards the outer flange section 360. A flex point is also defined between the bridge section 366 and the outer flange section 362. Of course these areas of flexibility are described as flex points when referring to any particular line through the seal as viewed in the Figs. However, since the seal is annular, the flex points or flex regions should be understood to exist around the entire circumference of the skirt section 362 as well as the entire circumference of the bridge section 366. The combination of all of these features allows the seal 36 to effectively accommodate a wider range of suction catheter diameters as compared to conventional seal designs.

In a particular embodiment, the outer flange section 360 has a radial thickness greater than a thickness of the conical skirt section 362. The outer flange section 360 may also have a radial thickness greater than the thickness of the bridge section 366. This configuration may be desired to enhance the flex action between the bridge section 366 and the outer flange section 362. In one such embodiment, as depicted in FIGS. 3A and 3B, the bridge section 366 and the conical skirt section 362 may have generally about the same thickness.

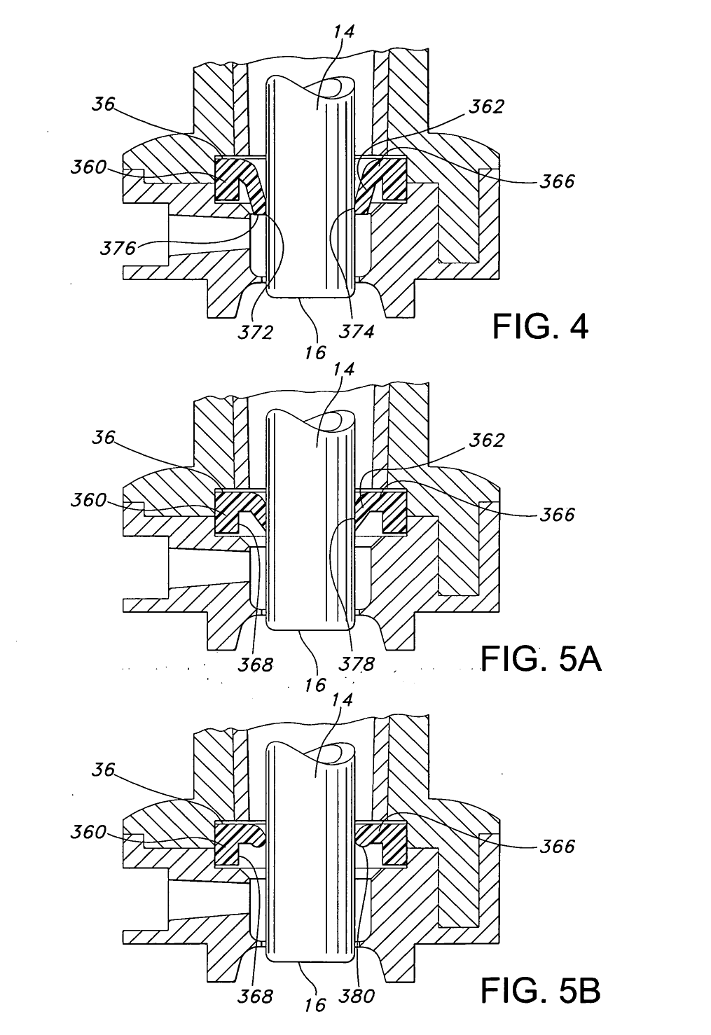

In another particularly unique embodiment, the outer flange section 360 may have a radial thickness greater than the thickness of the conical skirt section 362 and the bridge section 366. One such embodiment, shown in FIG. 4, may provide that the bridge section 366 has a thickness greater than the thickness of the conical skirt section 362.

In any of the aforementioned embodiments, the first angle between the bridge section 366 and the outer flange section 360 may vary. For example, in some embodiments, this angle may be about 90 degrees such that the bridge section 366 extends essentially perpendicular to the flange section 360. In alternate embodiments, the first angle may be greater than 90 degrees. The second angle between the bridge section 366 and the conical skirt section 362 may also vary, and may be a function of the first angle between the bridge section 366 and the outer flange section 360. For example, the second angle will generally be greater than 90 degrees when the first angle is 90 degrees or less, in some instances ranging from about 90 degrees to about 160 degrees. In other embodiments, the second angle may range from about 120 degrees to about 160 degrees. Alternatively, if the first angle is greater than 90 degrees, the second angle will generally be 90 degrees or less.

Although not a requirement, the bridge section 366 may be defined at a longitudinal end of the outer flange section 360 as shown in the Figs. However, in alternative embodiments, the outer flange section 360 may extend longitudinally above, below, or on each side of the bridge section 366.

The seal 36 may be made of various known resilient seal materials, with all of the seal sections 360, 362, and 366 being integrally formed into a single seal component 36. Alternatively, the seal sections 360, 362, and 366 may each be made of dissimilar materials joined by an adhesive or other method known to those of skill in the art. The seal lip 370 may also have various configurations, and may be directly formed with the conical skirt section 362 or subsequently defined at the end of the skirt section 362. In a particular embodiment, as shown in FIG. 4, the seal lip 370 may be a relatively sharp point edge 372 defined by angled planar surfaces 374 and 376 at the end of the conical skirt section 362. This configuration provides a point knife-edge engagement against the catheter surface. In an alternate embodiment, as shown in FIG. 5A, the seal lip is a planar surface 378 configured to engage the catheter 12 along a longitudinally extending plane. In still another embodiment, as shown in FIG. 5B, the seal lip 370 has a rounded edge 380.

It should be understood that the present invention includes various modifications that may be made to the embodiments of the respiratory apparatus described herein as come within the scope of the appended claims and their equivalents.

What is claimed is:

1. A respiratory apparatus, comprising:
a suction catheter having a tubular portion with a lumen defined therethrough, the suction catheter adapted for removing fluids from a patient by insertion of the tubular portion into an artificial airway of the patient and application of negative pressure to the lumen;
a manifold configured for connection in communication with the patient's artificial airway, the manifold including a port through which the suction catheter is advanced and withdrawn from the patient's artificial airway;
at least one seal disposed within the port around the suction catheter so as to provide a sliding frictional sealing fit with the suction catheter, the at least one seal formed from a resilient material and further comprising
a radially outer flange section, an inner conically shaped skirt section defining an aperture through which the suction catheter passes, and a bridge section between the outer flange section and the conical skirt section;
the outer flange section defining an inner diameter and the bridge section extending radially inward from the inner diameter at a first angle relative to the outer flange section;
the conical skirt section extending radially inward from the bridge section at a second angle relative to the bridge section that is less than the first angle; and
a seal lip defined at an end of the conical skirt section.

2. The respiratory apparatus as in claim 1, wherein the outer flange section has a radial thickness greater than a thickness of the conical skirt section.

3. The respiratory apparatus as in claim 2, wherein the outer flange section has a radial thickness greater than a thickness of the bridge section.

4. The respiratory apparatus as in claim 3, wherein the bridge section and the conical skirt section have a generally equal thickness.

5. The respiratory apparatus as in claim 1, wherein the outer flange section has a radial thickness greater than a thickness of the conical skirt section and the bridge section, and the bridge section has a thickness greater than a thickness of the conical skirt section.

6. The respiratory apparatus as in claim 1, wherein the first angle between the bridge section and the outer flange section is about 90 degrees.

7. The respiratory apparatus as in claim 1, wherein the first angle between the bridge section and the outer flange section is greater than 90 degrees.

8. The respiratory apparatus as in claim 1, wherein the second angle between the bridge section and the conical skirt section is greater than 90 degrees.

9. The respiratory apparatus as in claim 8, wherein the second angle is between about 120 and about 160 degrees.

10. The respiratory apparatus as in claim 1, wherein the first angle between the bridge section and the outer flange section is about 90 degrees, and the second angle between the bridge section and the conical skirt section is between about 120 and about 160 degrees.

11. The respiratory apparatus as in claim 1, wherein the bridge section is defined at a longitudinal end of the outer flange section.

12. The respiratory apparatus as in claim 1, wherein the seal sections are integrally formed from the same material.

13. The respiratory apparatus as in claim 1, wherein the seal lip comprises a sharp point edge defined by angled surfaces of the conical skirt section.

14. The respiratory apparatus as in claim 1, wherein the seal lip comprises a planar edge.

15. The respiratory apparatus as in claim 1, wherein the seal lip comprises a rounded edge.
